(19) United States
(12) Patent Application Publication
(10) Pub. No.: US 2002/0048750 A1
(43) Pub. Date: Apr. 25, 2002

(54) METHOD FOR THE PREPARATION OF A CHEMICAL LIBRARY

(76) Inventors: ANTHONY ROBERT CORLESS, MIDDLESEX (GB); DAVID ANDREW WENN, MIDDLESEX (GB); RICHARD EDEN SHUTE, MACCLESFIELD, CHESHIRE (GB)

Correspondence Address: PILLSBURY MADISON & SUTRO, 1100 NEW YORK AVENUE NW, NINTH FLOOR EAST TOWER, WASHINGTON, DC, 200053918

(21) Appl. No.: 09/403,227

(22) PCT Filed: Apr. 14, 1998
(86) PCT No.: PCT/GB98/01065

(30) Foreign Application Priority Data
Apr. 17, 1997 (GB) 9707743.2

(51) Int. Cl. C12Q 1/68; G01N 33/53; C07H 21/04; C07K 7/06; C07K 7/08

(52) U.S. Cl. 435/6; 435/7.1; 436/518; 436/523; 436/524; 436/525; 436/527; 436/528; 530/334; 530/335; 536/23.1

(57) ABSTRACT

A method for the preparation of a chemical library, which method comprises synthesizing the library on a plurality of individually coded synthesis particles and wherein the particles are tracked during library synthesis under the control of robotic apparatus.

Chemical libraries are a powerful way of providing compounds for the identification of active compounds in pharmaceutical, agrochemical and related industries.

Synthesis of the compounds on beads is a preferred method since it allows generation of diversity by the “split synthesis” method (Furka A., Abstr. 14th Int. Congr. Biochem., Prague, Czechoslovakia, 1988, 5, 47; Int. J. Pept. Prot. Res, 1991, 37, 487-493) and beads are convenient reaction supports which may be sequentially exposed to different reagents and washed with relative ease. Considerable research effort has taken place to find a reliable and simple method for the identification of active compounds from a library generated on beads. One promising approach has been to “tag” the bead at various stages of the synthesis, where each tag, or component of the tag, indicates the reagent(s) to which the bead has been exposed (K. D. Janda, Proc. Natl. Acad. Sci. USA, 1994, 91, 10779-10785). After testing, the tags associated with active beads are read and the chemical structures of the active compounds associated with those beads of interest is inferred. A particularly useful approach is the use of mixtures of halogenated aromatic compounds, incorporated in trace amounts at each stage of the synthesis, to form an identifiable (by gas chromatography) ‘binary code’ system for ligand definition (Borchardt and Still, J. Am. Chem. Soc., 1994, 116, 373-374).

However, chemical tags have certain limitations. For example, they can limit the choice of chemistry used to construct the library. Also, chemical tags can take significant amounts of time to read.

A further problem that has limited the use of tagged libraries to date is the essential requirement of handling individual synthesis beads. It is a property of the split synthesis method of combinatorial library synthesis that each bead carries one discrete chemical species. Consequently, at some point in the testing or screening protocols for the particular combinatorial library, the artisan who seeks activity of interest in a single library component must test single beads or the single library compounds that are associated with single beads. To date the handling of single, discrete beads, is still a significant difficulty in this process.

We have now found that technology developed and used in the microelectronics industry may be advantageously used in chemical library synthesis to provide positive control over the selection and movement of synthesis beads. In particular we disclose the use of robotic “pick and place” apparatus. We have found that such apparatus may be used to direct library synthesis with relative speed and precision.

Therefore in a further aspect of the invention we provide a method for the preparation of a chemical library, which method comprises synthesising the library on a plurality of individually coded synthesis particles and wherein the particles are selected according their individual codes and manipulated during library synthesis under the control of robotic apparatus.

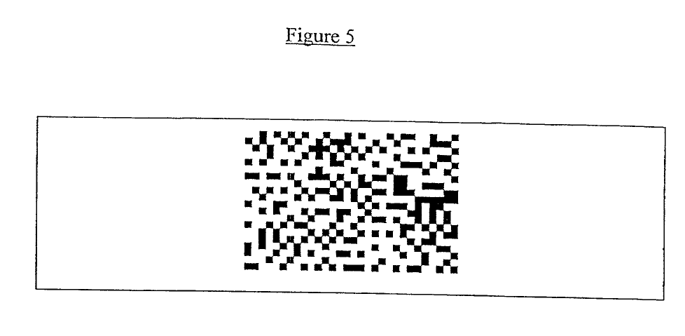

By “individually coded synthesis particles” we mean synthesis particles comprising an individual tag or tags. Convenient synthesis particles will be apparent to the scientist of ordinary skill. The tag(s) may be present on the synthesis particles in the form of a physical code. Examples of convenient tag(s) and/or codes are disclosed in our UK patent applications entitled “Method”, “Methods” and “Process” filed Apr. 17, 1997. These UK patent applications nos. 9707744.0, 9707742.4, 9707741.6 respectively and their contents are herein incorporated by reference. A preferred code is provided by a 2-dimensional bar code. This is an arrangement of dots or patches in which the position of a mark in both the x and y axes is significant. An example is shown in FIG. 5. These codes have been developed in response to a number of requirements for product marking, e.g. tracking packages in shipment (Maxicode), machine readable bill-of-loading (PDF417), coding of pharmaceutical containers (Snowflake). Taking the Snowflake code as a good example of what is possible, this code carries an information content equivalent to 100 numeric digits in an area 5 mm×5 mm. The code is readable in any orientation, and with a high degree of error correction; up to 40% of the code can be affected by debris or distortion and the data content can still be fully recovered. The printed density of the Snowflake code quoted is applicable to a code designed to be applied by ink-jet printing, punched holes, laser marking etc. With a more sophisticated writing process the data density of a 2-D bar code may be improved considerably.

The synthesis particles are conveniently of up to 5 mm, such as up to 2 mm, for example up to 1 mm in their largest dimension.

The robotic apparatus is preferably a robotic “pick-and-place” machine. Such machines are used in the microelectronics industry, most commonly in the placement of surface mount components. However we can find no suggestion of their use in other technical areas. In the microelectronics industry, the components to be manipulated are typically resistors and capacitors taking the form of flat, ceramic parts—variously known as 0402, 0805, 1206 etc., with resistive components and individual diodes also being available in a cylindrical form known as MELF or micro-MELF—and active devices such as individual diodes and transistors taking the form of plastic moulded packages such as SOT-23, integrated circuits packages such as SO-8, BGA and so on. These devices are of small size, with the 0402 device being only, approximately, 1 mm×0.5 mm in extent. The industry has developed robots able to manipulate these packages at high-speed with precise placement to predefined locations on, for example, a printed circuit board. With the increasing sophistication of the devices to be manipulated, both in terms of the number of connections and the reduction in the size of individual connections and spacing between connections, the industry has adopted the use of video cameras and pattern recognition systems to ensure the accurate placement of complex components. Machines which are commonly employed in the microelectronics industry use a vacuum pick up to hold the components. The speed of placement varies with the mix of components and the size of the board onto which they are to be placed. However, the quoted throughput is commonly in the range of a few hundred to perhaps 30,000 components placed per hour. In an application such as the present, a mid-range throughput of perhaps 1 bead per second might be achieved by machines typical of those currently available.

In the conventional use of a pick-and-place machine the components are moved from pre-defined “feeder” positions to pre-defined locations on the printed circuit board. The camera and pattern recognition system being employed only to enhance the quality of the performance of the task. In the present application, the identity of the particle will be read by means of the camera, this identity being used in conjunction with the control software to determine the locus to which the particle should be moved. The general nature of the modifications to the software and machine operation will be apparent to those appropriately skilled.

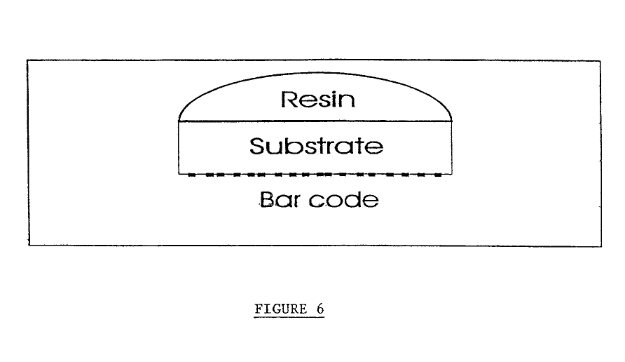

The robotic pick and place apparatus may, for example, be used to pick up the particles in turn before each reaction step, present them appropriately to the code reader with the code marks being read using a CCD camera through suitable optics. The bead would then be identified by means of the code and the pick up tool would then direct and deposit the bead into the appropriate reaction vessel or locus ready for the next stage of the library synthesis and assay process. Furthermore, beads that possess the general structure as described in FIG. 6, in that they possess one face that is flat and carries the bar code, could readily be made to lie on one face, thus facilitating pick-up by the robotic tool. Manipulation by the pick-up tool is further facilitated by arraying the beads on a flat plate such that the codes of many beads can be read by a CCD camera simultaneously, thus expediting the directing and recording process. Alternatively, a plate with suitable indentations may be produced that allows the beads to settle in an orientation of choice, for example flat, code-bearing side upwards, to aid imaging and recording of the bar code.

By way of further example, the “composite synthesis particles” are individually picked-up, and the bar code read using the camera and pattern recognition system. The control software determines, from its record of the prior chemical history of the synthesis particles, an appropriate location at which a particle should be deposited. The determination of particle positioning may be under the control of an algorithm which will seek to maximise the throughput of the machine, for example by minimising the total movement of the placement head. The nature of the modifications required to the control software to implement such function will be apparent to those appropriately skilled.

It can be seen that the requirements on the code reading camera are straightforward. This is shown by considering, by way of example only, the details of a useful code. For even large libraries of compounds it is unlikely that there will be a need for more than perhaps 2³² (>4,000,000,000) codes. To allow so many codes we only require for example 32 dot positions on the 2-D bar code. However, it is preferable to allow a far larger number of positions so that the code can have the beneficial features of orientation independent reading, and substantial error correction. Hence we may require a code with up to 256 dot positions, for example in the form of a 16×16 matrix. If we further assume by way of example only that this matrix will occupy the central 800 μm square section of a 1 mm square particle, then the side length of a code pixel is 50 μm. This is large compared to the resolution available with optical elements. Typically CCD devices have pixel pitches of order 10-25 μm hence it can be seen that it is readily possible to image the code mark so that each pixel of the code fills several pixels of the CCD. CDD cameras having pixel arrays of 256×256 pixels are commonly available, with single devices having as many as 4096×4096 pixels being reported for astronomical use. Hence it will be apparent that there is little difficulty in forming an image of the code which can be resolved by a typical CCD element with high precision allowing the code to be read by image processing software.

One preferred embodiment is the use of a single coded particle for each target library compound required. While the example below discusses a case in which the number of divisions is equal at each chemical process step, this is merely a convenient and common practice. It is not an essential feature of either combinatorial chemistry or the present invention.

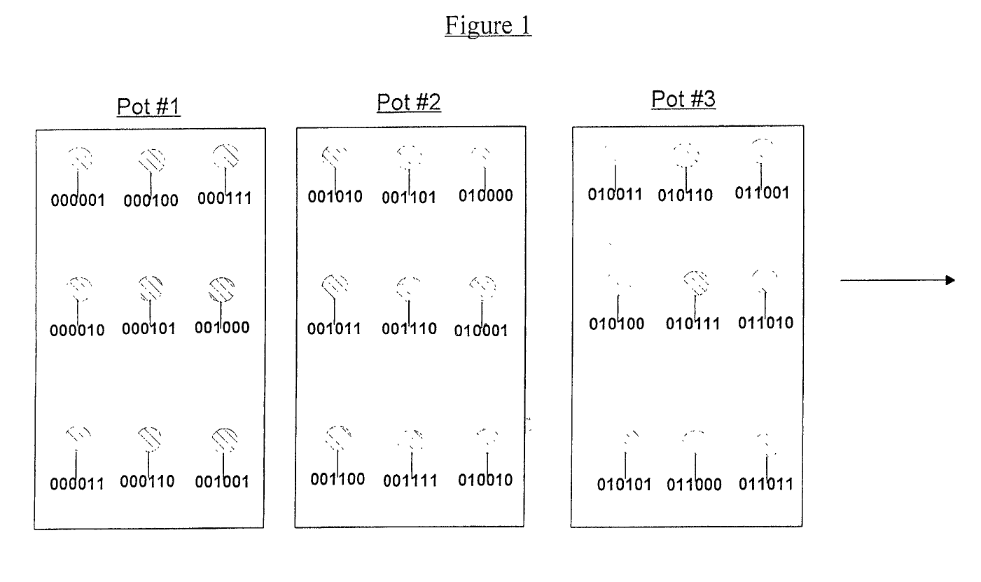

For example, consider a library of, say, 27,000 compounds, which is to be made by a tracked split-synthesis process using 30 primary diversity substituents, or building blocks, 30 secondary building blocks and 30 tertiary building blocks (ie. a 30×30×30 library). One would start with 27,000 unique composite synthesis particles. Into each of the 30 primary reaction vessels would be tracked 900 synthesis particles. The first stage of the library synthesis would be performed, and the particles would be recovered then deposited and tracked into secondary reaction vessels such that no more than 30 particles that were in any primary reaction vessel are placed in the same secondary reaction vessel. The second stage of the library synthesis would then be performed, and again the particles would be recovered, deposited and tracked into tertiary reaction vessels. This time, however, it is an essential part of the placement process that no two particles that have been in the same primary and secondary reaction vessels are deposited in the same tertiary vessel. The third and final stage of library synthesis would then be performed, and again the particles would be recovered. Provided no two particles have passed through the same three reaction vessels, and there have been no particle losses, there will be 27,000 distinct compounds attached to 27,000 composite synthesis particles. A simplified version of the above process is described (in FIGS. 1-4) for a library of 27 discrete compounds, 27 discrete coded synthesis particles, 3 primary ‘A’ building blocks, 3 secondary ‘B’ building blocks, and 3 tertiary ‘C’ building blocks. Such a directed process thus involves the active steering of the discrete beads down code-specific and, for any one bead in the defined library, unique, process paths resulting in a single compound per coded particle. It is a process that is considerably assisted by the use of robotic pick-up tools. The association between each code and chemical process sequence may be defined in advance or assigned dynamically as part of a routine to optimise the throughput of the equipment. A further advantage to the use of a pick-and-place machine in this process is that at the end of the synthesis it allows, if desired, for the convenient selection of one or more defined subsets of the library for special treatment or further processing. This aspect of the method provides a further key embodiment to the method of the invention.

A second preferred embodiment of the method involves a random or stochastic synthesis. Here a large number of beads, say 3 or 4 or more particles per target compound, would be used, thus removing the need for an active steer for each and every particle at each reaction stage. In a preferred embodiment of this process for the same 27,000 member compound library, where 3 particles are used per target compound, the initial 81,000 (3×30×30×30) bar coded composite particles would be divided randomly into the 30 primary reaction vessels and the first stage of the library synthesis performed. The particles would be recorded either immediately before the first stage of the library synthesis or following this stage. They may then be either mixed and split again randomly into 30 groups, or a one thirtieth portion of each of the groups of particles from a primary reaction vessel may be combined to produce each of the 30 groups of particles for the second synthesis stage. At each reading stage the beads in each group would, in turn, be spread over a flat or indented plate. The plate would be mounted on an x-y stage and scanned under a camera system. This would read all of the bar codes and thereby record which beads were to go through the particular synthesis step. Alternatively, the composite particle would be designed to allow the code to be read from either side, or a transparent tray would be employed with a camera viewing each face of the scanned tray. This process would be repeated, as necessary, for each of the three stages of library synthesis. The synthesis particles may or may not be mixed at the end of the synthesis. If they are not, then information about the last reaction vessel can be retained over and above the information stored by association with the bar code. If they are mixed at the end of the synthesis, then introduction of a pick-and place machine, as used for the directed synthesis, would allow a full set, or defined subsets of unique beads to be extracted following the random synthesis. Alternatively, even if they are not mixed, the particles from each of the last reaction vessels can be spread out over the flat or indented plate and, using the pick-and-place machine, defined subsets extracted.

The plates used to support the beads during the reading (recording) stage may take the form of essentially flat plates with the beads dispersed at random, or they may be trays with wells or pockets to hold individual beads—such as the waffle trays familiar as a transport and storage means for semiconductor die. The particles will tend naturally to lie on one or other major face, and can be encouraged so to do by application of slight vibration to the tray. The particles may be designed such that the code mark is readable whichever major face is upright, eg a transparent particle, or the tray may be designed to be transparent with upper and lower cameras being employed to read the codes. Alternately, the beads which are the correct face uppermost may be read, a second tray abutted over the first and the whole inverted—thereby inverting the beads and allowing reading of those code marks which were previously obscured. Alternately, a ‘die flipper’ such as is used in the preparation for flip chip alignment and bonding, in the microelectronic industry, may be used to invert selected, individual beads.

On conclusion of the chemical library synthesis using coded particles, but prior to optional cleavage of the compounds from the particles, the manipulative robotic device picks up each coded particle in turn, presents it appropriately to the code reader and the code marks are read. The device then deposits the individual particles to individual vessels in readiness either for direct assaying of individual compounds attached to individual particles, or for removal of the compounds from the particles by a cleavage process, and assaying of the compounds in free solution. Alternatively, the tool picks up each coded particle and deposits the individual particles to individual vessels in preparation either for on-particle screening or cleavage, but without the code on the particle being read. In the latter case, particle code-reading is not done until activity of interest is associated with a particular vessel, at which point the particle of interest is recovered, a process which may or may not involve use of the pick-up tool, the code is read and this allows the chemical structure of interest to be inferred. By vessel, we intend and include an array of linked vessels such as a “waffle tray”, or other similar tray including micro-titre plates and modifications thereof, which may be used to faciliate handling of groups of compounds and supply a spatial reference thereto. In particular, the use of arrangements of linked vessels which facilitate the screening and readout processes is intended and included.

The use of a flat or indented plate to array the coded particles and a CCD camera to read the codes of the arrayed particles as described above, as well as selective use of robotic pick-up tools to manipulate coded particles at any stage of a combinatorial library synthesis and screening process, for example only at the end of the combinatorial library generation process, are further novel aspects to this invention. However, it should be noted that the processes outlined above are intended to be illustrative and by no means limit those procedures that might be followed using coded particles, flat or indented plates for particle arraying, and robotic pick-up tools.

It should be noted that subject to practical considerations, synthesis of compound libraries on the coded particles of the invention may comprise any convenient number of individual reaction steps. For example library synthesis may comprise 2, 3, 4, 5, 6 or more reaction steps.

The chemical libraries prepared using the methods of the invention may comprise any convenient number of individual members, for example tens to hundreds to thousands to millions etc., of suitable compounds, for example peptides, peptoids and other oligomeric compounds (cyclic or linear), and template-based smaller molecules, for example benzodiazepines, hydantoins, biaryls, carbacyclic and polycyclic compounds (eg. naphthalenes, phenothiazines, acridines, steroids etc.), carbohydrate and amino acids derivatives, dihydropyridines, benzhydryls and heterocycles (eg. triazines, indoles, thiazolidines etc.). The numbers quoted and the types of compounds listed are illustrative, but not limiting.

Preferred compounds are chemical compounds of low molecular weight and potential therapeutic or otherwise biologically active agents—such as pesticides. They are for example of less than about 1000 daltons, such as less than 800, 600 or 400 daltons.

Any convenient biological of interest such as a receptor, enzyme or the like may be contacted with the chemical library as above in an assay or test system apparent to the scientist of ordinary skill.

Advantages of the use of manipulative robotic devices such as pick-and-place machines in combinatorial chemistry include: the ability to form an essentially complete library consisting of a single composite synthesis particle per chemistry either by selection from a larger stochastically formed set or by manipulation of particles at all stages; the ability to select a sub-library of controlled diversity for an initial screen which is designed to highlight the ‘volumes of chemical space’ in which compounds of interest are to be found, in particular the ability to decide not to select individual particles or sub-libraries of particles—followed by a subsequent selection of further sub-libraries surrounding the regions of interest, without further chemical synthesis processes being required. In this way, the technique significantly enhances the throughput of the overall drug discovery process.

Advantages of the libraries of the this invention include:

(i) the coded particles are generated prior to any chemical synthesis being undertaken, so no time is taken up introducing tag information during the library synthesis process;

(ii) the coded particles may be rapidly read and checked before any synthesis is undertaken, any beads carrying unreadable codes can be rejected, thus allowing the in-process bead reading to be of even higher fidelity;

(iii) close coupling between the tag and the chemistry allows tracking at all stages eg. through all process steps, library storage and screening steps.

(iv) the ability to select subsets of the library of controlled diversity to minimise the number of compounds screened.

In summary, the use of a coded particle to assist in bead identification and tracking and a camera and manipulative robotic pick-up tool to assist in the tracking and deposition of any particular labelled bead through each stage of a split-synthesis, one-compound-one-bead library generation process, one can conveniently associate, at the end of the process, a single chemical structure with a single code. The ability to utilise manipulative robotic devices to assist in the establishment of that association has considerable utility for the assaying and deconvoluting, or decoding of compound libraries.

The invention will now be illustrated but not limited by reference to the following Figures wherein:

FIGS. 1-4 show the processes involved in the generation of a model, tagged library of 27 discrete compounds on 27 discrete beads. The 27 discrete beads each carry a unique tag, in this case indicated by a 6-bit binary code, which numbers the beads from 1 to 27.

FIG. 1 shows the 27 discrete beads in pots #1, #2 and #3 prior to application of chemistry “A”.

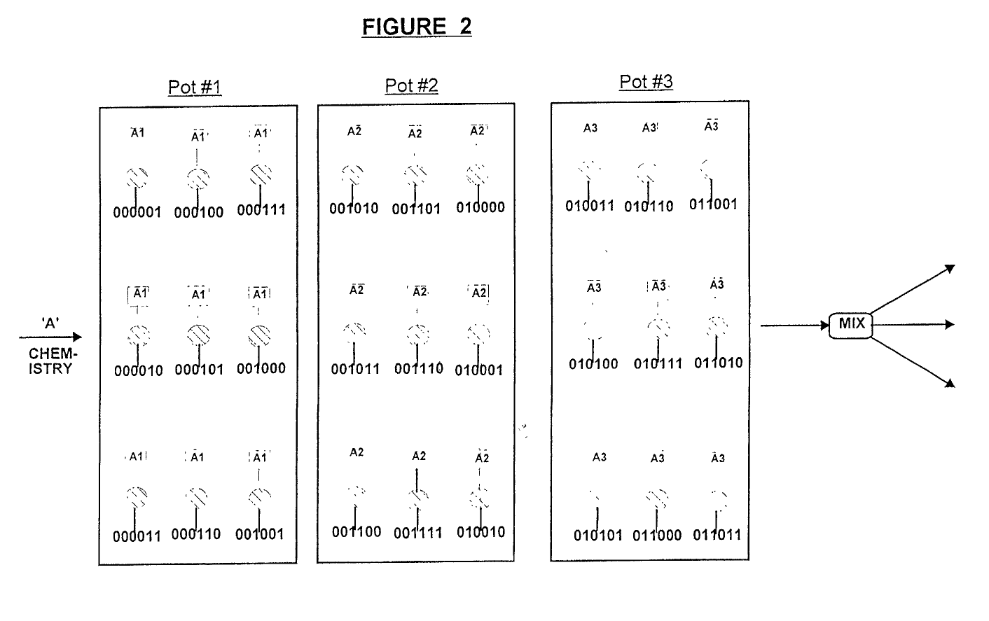

FIG. 2 shows the application of chemistry “A” to the library and subsequent mixing of the contents of pots #1, #2, and #3. The resulting mixture is divided into the three pots.

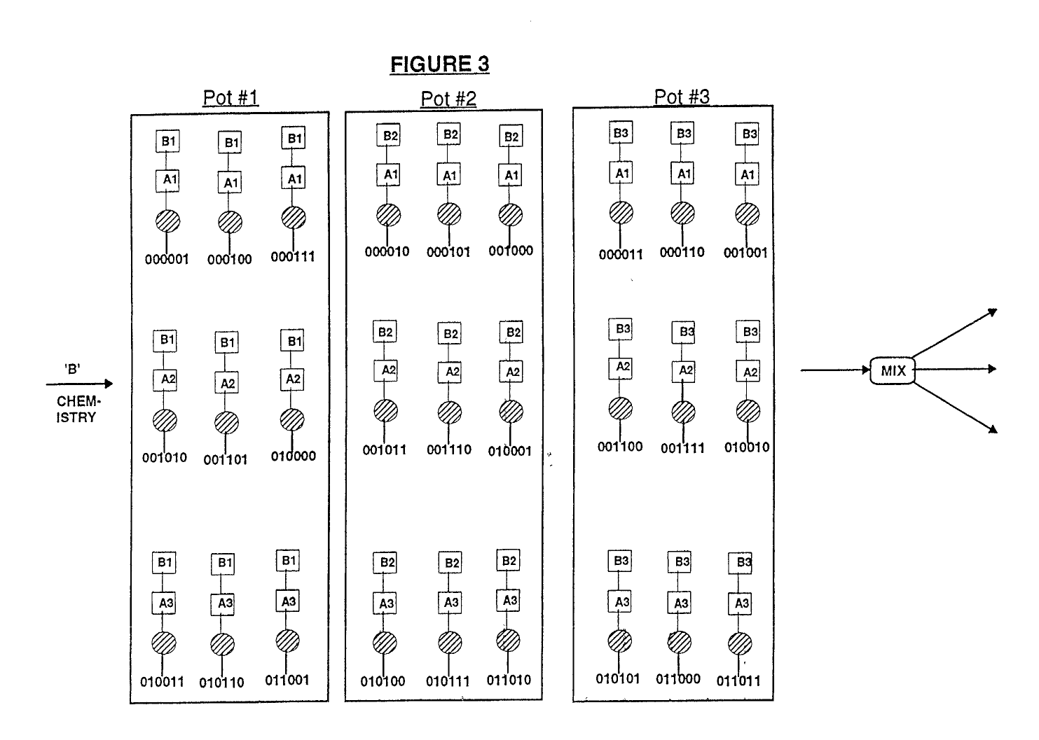

FIG. 3 shows the application of chemistry “B” to the library and subsequent mixing of the contents of pots #1, #2, and #3. The resulting mixture is divided into three pots.

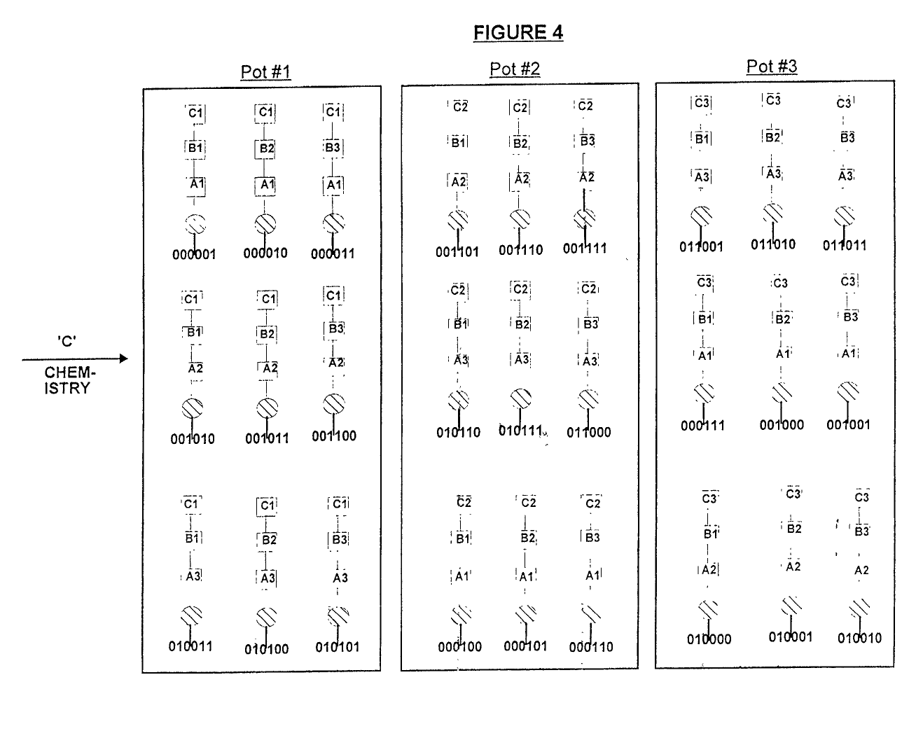

FIG. 4 shows the application of chemistry “C” to the library and the library compounds so obtained.

The diversity elements introduced during the various (‘A’, ‘B’ and ‘C’) chemistry processes are indicated by the boxed indicators (A1, A2, B3, C3 etc.) which are attached to the hatched circles, which in turn represent the synthesis particles. The term ‘MIX’ includes both the recombining of the 27 particles and their redistribution into the 3 further pots, or reaction vessels in preparation for the next stage in the library synthesis.

FIG. 5 shows an example of a 2-dimensional bar code.

FIG. 6 shows a flat bead comprising a 2-dimensional bar code.

1. A method for the preparation of a chemical library, which method comprises synthesising the library on a plurality of individually coded synthesis particles and wherein the particles are selected according their individual codes and manipulated during library synthesis under the control of robotic apparatus.

2. A method as claimed in claim 1 wherein the individual codes are provided by 2-dimensional bar-codes.

3. A method as claimed in claim 1 wherein the robotic apparatus is a robotic “pick and place” machine.

4. A method as claimed in any one of the previous claims wherein the robotic apparatus presents the synthesis particles in turn to a code reader prior to, following or between chemical synthesis steps.

5. A method as claimed in any one of the previous claims wherein the synthesis particles have a substantially flat surface on which the individual code is provided.

6. A method as claimed in any one of the previous claims wherein the synthesis particles are spread over a plate or tray prior to code reading and robotic manipulation.

7. A method as claimed in claim 6 wherein the plate or tray is provided with indentations/formations so as to allow the particles to be held in an orientation of choice thereby facilitating simultaneous reading of individual codes prior to robotic manipulation.

8. A method as claimed in claim 6 or claim 7 wherein all or a part of the synthesis particles are inverted on the plate or tray for code reading.

9. A method as claimed in any one of claims 6-8 wherein the plate or tray is transparent.

10. A method as claimed in claim 1 wherein the particles comprise individual bar-codes and are selected and manipulated by a robotic “pick and place” machine linked to an optical bar-code reader.

11. Use of a chemical library prepared according to any one of the previous claims in screening methods to identify compounds which modulate the activity of a biological of interest.

12. The use as claimed in claim 11 wherein robotic apparatus is used to select library members on a random or directed basis for use in the screening methods.

13. The use as claimed in claim 12 wherein the directed basis is a structure/activity relationship.

14. Chemical library synthesis/analysis apparatus which comprises a robotic “pick and place” machine adapted to read and manipulate individually coded synthesis particles.
